(19) United States
(12) Patent Application Publication
(10) Pub. No.: US 2018/0268102 A1
(43) Pub. Date: Sep. 20, 2018

(54) PHASING ANALYSIS WITH DYNAMIC PROGRAMMING ALGORITHM

(71) Applicant: Sirona Genomics, Inc., Mountain View, CA (US)

(72) Inventors: Ming LI, Mountain View, CA (US); Chunlin WANG, Mountain View, CA (US)

(73) Assignee: Sirona Genomics, Inc., Mountain View, CA (US)

(21) Appl. No.: 15/764,132

(22) PCT Filed: Sep. 28, 2016
(86) PCT No.: PCT/US16/54171

§ 371 Date: Mar. 28, 2018

Related U.S. Application Data
(60) Provisional application No. 62/233,699, filed on Sep. 28, 2015.
(60) Provisional application No. 62/284,356, filed on Sep. 28, 2015.
(60) Provisional application No. 62/399,707, filed on Sep. 26, 2016.

(51) Int. Cl. G06F 19/22; C12Q 1/6869; G06F 19/18

(52) U.S. Cl. CPC G06F 19/22; G06F 19/18; C12Q 1/6869

(57) ABSTRACT

Provided herein are methods and systems useful for the determination and assignment of haplotypes for genetic loci.

### TECHNICAL FIELD

The presently disclosed subject matter relates generally to genotyping and assigning haplotypes to genetic loci from organisms. In particular, the invention provides systems and methods for assigning haplotypes and determining genotypes in human leukocyte antigen (HLA) genes from next generation sequencing (NGS) data.

### BACKGROUND

With the advent of next-generation sequencing, the processing of a large amount of genotyping data is a complicated task. On a typical NGS platform, a vast number of gene fragments are sequenced in high-throughput and each position on a genome can be sequenced repeatedly in different fragments containing overlapping regions, resulting in multiple reads per position. The number of reads per position, commonly known as the coverage, can range from tens to hundreds or even more in a typical sequencing run although there exists a significant variation from gene to gene and sample to sample. The sequenced fragments are then assembled and mapped to the genome using computational methods in order to obtain the full sequence of the target gene or region.

Each of the sequence reads from the NGS comes from a single chromosome. However, information about which chromosome a sequence read comes from is not known a priori from the sequence read data. Using computational methods, the two haplotypes spanning multiple polymorphic loci may be obtained from the NGS data by joining reads that share alleles at common variants. The problem of resolving phased haplotypes from NGS sequence reads may be known as “haplotype assembly” or “phasing analysis.” When the phases of the sequence reads are resolved, the output of the target gene sequence may be in the form of two phased, partial haplotypes, or multiple phased partial haplotype pairs at different loci.

The presence of polymorphisms in various alleles provides a significant challenge in deconvoluting sequence data from a tremendous amount of fragments and trying to assemble an allele from such data. Previous methods primarily rely on consensus reference genome at a polymorphic site to establish a particular haplotype. However, in highly related gene families, such an approach is error-prone and leads to a number of false positives because of the high-degree of similarities in the sequence of the gene variants and incomplete or inaccurate information about the consensus reference genome sequences. Further complicating matters is the presence of pseudogenes that may interfere with the haplotype determination because these pseudogenes share common ancestry with functional genes and have very similar sequences to those functional genes. Additionally, sequencing errors significantly increase the difficulties of phasing analysis in regions with a high degree of polymorphism. Typically, the phasing analysis starts with sequence alignment to a reference genome which may itself be incomplete and/or contain errors especially in complicated genomic regions. Sequencing and alignment errors are thus retained throughout the entire phasing analysis process and result in undesired inaccuracies.

One such gene family that requires an improved method of haplotype determination is the human HLA gene family. The human HLA genes span a region of about 3.6 Mb and are the most polymorphic in the human genome. The human HLA genes share extensive similarities with each other and many pairs of alleles are different only slightly, for example, by a single nucleotide. However, human HLA genes play a key role in immune responses and minor differences in the HLA gene sequence can have a significant impact on many clinical indications, for example, the specificity of antigen presentation, histocompatibility in transplantation, autoimmunity, etc. Accordingly, there is a great need for processing methods that correctly identify and assign alleles from sequence data corresponding to highly related, complicated genomic regions, such as the human HLA genes.

### SUMMARY

In some embodiments, the present invention provides a method for assigning a partial haplotype to a genetic locus comprising: (a) providing sequence reads for said genetic locus; (b) processing said sequence reads into an assembly read, (c) generating a consensus sequence from said assembly read comprising only polymorphic sites within said genetic locus to obtain a polyread; (d) constructing a scoring matrix by converting said polyread into a binary string; (e) processing said scoring matrix by generating a score that minimizes the total number of discrepancies between the consensus sequence and said sequence reads at only said polymorphic sites; and (f) assigning said partial haplotype to said genetic locus by reconstructing said locus using the score from step (e).

The sequence reads manipulated by the present invention can be in the form of paired-end sequence reads or single-end reads. An example of the former includes those reads generated by paired-end sequencing technology such as the one currently in use on Illumina® based platforms, including the MiniSeq®, the MiSeq®, the NextSeq®, and the HiSeq® series of instruments. In alternative embodiments, the sequence reads processed by the invention may be generated by any other suitable sequencing methods, including, but not limited to Sanger-based sequencing, Ion-torrent based sequencing, pyrosequencing technologies, single molecule based sequencing technologies, and the like.

In certain embodiments, when paired-end reads are used, the two paired-ends for the same gene fragment may be first combined to produce a single sequence that is used for further processing. Gaps are filled between the two ends when necessary. The advantage of paired-end reads is that such data typically provide better quality control when the two ends are combined. In other embodiments, paired-end reads are used separately as single-end reads from each end.

The methods of the invention also provide a de novo assembly of short sequence reads to generate a long, contiguous read assembly against which the sequence reads are aligned for phasing analysis and SNP calling for each position may be performed for assessing sequencing errors. The objective of the de novo assembly is to reproduce a genome sequence which is likely the source of all the sequence reads. A number of heuristic de novo assembly algorithms may be used in the present invention.

In certain embodiments, after the de novo assembly is built, the underlying genome may be used for assessing sequencing errors on the sequence reads using Bayesian estimates. In the Bayesian estimates, the prior distribution of the nucleotides may be determined from established reference sequences for the genomic region. As such, insights from known polymorphism and linkage disequilibrium in the target gene region may be used to assess the data quality and guide the phasing analysis. Further, Bayesian estimates may be used for identifying new alleles in the genetic locus.

In certain embodiments, the process of de novo assembly may be repeated after Bayesian estimates on the sequence reads are obtained and erroneous or unreliable reads are eliminated.

In preferred embodiments, the sequence read information is manipulated to identify polymorphic sites within the reads. After identification, the method processes the polymorphic site information into a polyread which excludes the conserved sequences that are present in the sequence reads and only retains the polymorphic site information. Further, the sequence read information is converted into a binary string comprising a value of 0 or 1 for each polymorphic site within said polyread. In some instances, the polyread is represented as Xᵢϵ{0,1,−}ⁿ, where “−” is a gap indicating that the position is not covered by the polyread. Some polyreads may not comprise a gap at either end of the polyread. While the values 0 and 1 are typically used for representing a binary string, other types of binary representation of the polyread can also be used.

In certain embodiments, the invention provide a scoring matrix is represented by a k-mer of said binary string and s(i,r) as the score for a partial haplotype starting at position 0 and ending at position i+k−1 with suffix r and methods for generating said scoring matrix. Further, scoring matrix may also be represented by

s(i,r)=min_{b=0,1}(b=0,1)[(s(i−1,(b,r[0,k−2]))+h(i,r))]],

where b is a binary number of 0 or 1, r[0,k−2] is the length k−1 prefix of r, (b,r[0,k−2]) is a k-mer binary string generated by concatenating b with r[0,k−2], h(i,r) is the minimum of the total number of discrepancies between r or the complement of r and all reads starting at position i. In some embodiments, r may comprise either r or the complement of r.

Methods of the invention also provide for the determination of the minimum discrepancy of the polyread to the sequence reads. As such, the result for s(i,r) represents the minimum discrepancy. In determining the minimum discrepancy, s(i,r) excludes gaps in the polyread. Such minimum discrepancy values are used in the methods of the invention to analyze the polyreads to generate said partial haplotype. Accordingly, methods of the invention include iteratively generating a partial haplotype of the genetic locus represented by the minimal value of s(n−k,r) over all r. In some embodiments, the methods may further comprise where s(n−k−1,(b,rₙ[0,k−2])), where b is the recorded symbol for computing s(n−k,rₙ).

In certain embodiments, the objective function to obtain minimum discrepancy uses a weighted scoring scheme based on the quality of the sequence reads. The weighted scoring scheme applies a weight in accordance with the quality of sequence reads at each position and corrects for the errors introduced during the sequencing process.

To assemble a larger haplotype of the genetic locus beyond the coverage of a specific set of sequence reads, the invention provides for methods of generating a complete haplotype for a genetic locus by sequentially processing and assembling partial haplotypes for the locus as generated by the complementary methods outlined in the invention.

In certain embodiments, the invention provides for assessing the minor allele frequency representing the frequency of the second most abundant base at a particular position in the locus. The method then employs a threshold cutoff of the minor allele frequency to further refine and improve the haplotype assignment at a particular base in the locus.

The methods of the invention are useful in haplotype determination where the genetic locus has at least 5, at least 10, at least 15, at least 25, at least 50, at least 75, at least 100 or even more polymorphic sites.

The methods of the invention are preferably performed on a digital computer. The computer may comprise a processor, digital storage medium and memory for storing the output for the method. Further, the method may be performed on the digital computer with input of sequence information from a database, another computer, or manually. The output of the method may also be transmitted to another computer, further processed by another method or presented to the user in an appropriate interface.

In certain embodiments, the present invention provides methods for processing sequence reads from NGS data and determining haplotypes in the human HLA genes. In some embodiments, the genetic locus of the human HLA genes for which haplotypes are phased is selected from the group consisting of HLA-A, HLA-B, HLA-C, HLA-DRB1, HLA-DRB3, HLA-DRB4, HLA-DRB5, HLA-DQB1, HLA-DQA1, HLA-DPB1, and HLA-DPA1. In certain embodiments the HLA-DRB3, HLA-DRB4 and HLA-DRB5 together are considered one locus.

Other features, advantages, and implementations of the present disclosures, not expressly disclosed herein, will be apparent to one of ordinary skills in the art upon examination of the following detailed description and accompanying drawings. It is intended that such implied implementations of the present disclosure be included herein.

### BRIEF DESCRIPTION OF THE DRAWINGS

FIG. 1A schematically illustrates the unknown arrangement of polymorphic sites as they relate to either the copy 1 or copy 2 of the genetic locus.

FIG. 1B schematically illustrates the identification of polymorphism and assignment of phased haplotypes corresponding to copy 1 and copy 2 of the genetic locus using the methods of the invention.

FIG. 2 schematically illustrates the partition of the genetic locus into multiple subsets for resolving partial haplotypes in each subset.

FIG. 3 schematically illustrates a workflow employed by an embodiment of the invention for phasing analysis and identification of genotypes in the human HLA genes.

### DETAILED DESCRIPTION

Next generation sequencing (NGS) is a technology that is used to sequence a number of distinct DNA sequences simultaneously. In one approach, NGS is used to sequence a number of genes or gene fragments from multiple samples or individuals in one run. Once the sequence information for that run is generated, the information, representing millions of reads per sample is assigned to one sample according to a barcode sequence or other identifying marker for each sample.

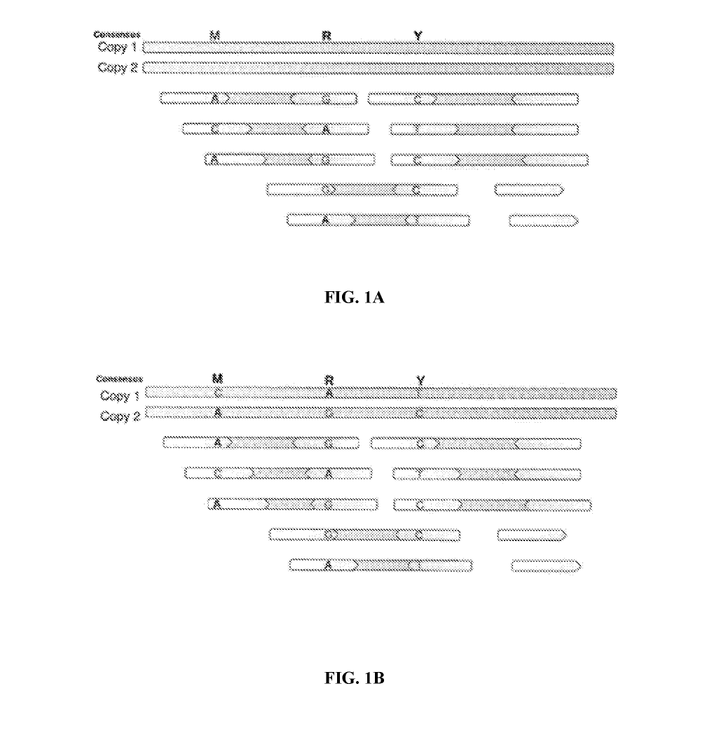

The present invention provides methods and systems for determining haplotypes from a large number of sequence reads using a suitable sequencing technology. Referring to FIG. 1A, the smaller fragments represent unassigned consensus sequences containing polymorphic sites that correspond to positions M, R, and Y. What is unknown from the sequence reads is the arrangement of bases at each position in either copy 1 or copy 2. In FIG. 1B, the identified polymorphisms at positions M, R, and Y can be determined and assigned for copy 1 and copy 2 using the methods of the invention.

The sequence reads suitable for the present invention can be in the form of paired-end sequence reads or single-end reads. Examples of such data include those generated on an Illumina platform, such as the MiniSeq®, the MiSeq®, the NextSeq®, and the HiSeq® series of instruments. In alternative embodiments, the sequence reads processed by the invention may be generated by any other suitable sequencing methods, including, but not limited to Sanger-based sequencing, Ion-torrent based sequencing, pyrosequencing technologies, single molecule based sequencing technologies, and the like.

In certain embodiments, when paired-end reads are used, the two paired-ends for the same gene fragment may be first combined to produce a single sequence that is used for further processing. Gaps are filled between the two ends when necessary. The advantage of paired-end reads is that such data typically provide better quality control when the two ends are combined. In other embodiments, paired-end reads are used separately as single-end reads from each end.

In the present invention, the sequence reads are used to build a de novo assembly representing the underlying genome sequence. The methods of the invention provide a de novo assembly of short sequence reads to generate a long, contiguous read assembly against which the sequence reads are aligned and SNP calling for each position is performed. The objective of the de novo assembly is to reproduce a genome sequence which is likely the source of all the sequence reads. A number of heuristic de novo assembly algorithms may be used. For example, the De Bruijn k-mer graphs may be used for building an assembly with shorter reads. For another example, when longer reads data are obtained, the de novo assembly can be built using sequence overlaps and clustering algorithms. Generally, the de novo assembly algorithm first creates simple contig sequences using all the sequence reads. Next, all the reads are mapped using the simple contig sequence as reference such that the genome assembly is as short as possible and includes as much of the input data as possible.

Knowledge about the underlying genome region may be used for the de novo assembly process. Such knowledge may include, for example, identification and counting of highly conserved sequence regions, identification of heterozygous SNPs at given positions, identification of repeat regions, etc. Further, data quality measurements may be used to guide the de novo assembly. For example, if low quality regions are detected, these sequence reads may be trimmed before the de novo assembly. When paired-end data are obtained, the paired-end reads are expected to map onto the genome assembly with a given respective orientation and a given distance from each other.

De novo assembly is particularly useful in the human HLA genes due to a high degree of similarities and polymorphisms among variants of the HLA genes. De novo assembly places the emphasis on sequencing data obtained from the assayed samples and incorporates knowledge about the target genome sequences. As such, de novo assembly is amenable to known variations in the genomic region as well as new alleles not reported in the literature. To the contrary, previously disclosed methods rely on a reference genome in performing alignment. Such methods suffer from the incompleteness and inaccuracies from available human HLA gene databases and those inaccuracies will be carried over to the haplotype phasing steps.

In certain embodiments, after the de novo assembly is built, the underlying genome may be used for assessing basecall on the sequence reads using Bayesian estimates. In human HLA genes, assessing basecall is particularly useful because it helps identify sequencing errors versus novel alleles. Bayesian estimates on the sequence data can be obtained using prior distributions that incorporate known linkage disequilibrium information in the genomic region of the HLA genes. Sequence reads that deviate significantly from estimated sequences based on LD information in adjacent loci may be flagged as erroneous or unreliable. Bayesian estimates provide a quality check on the sequence reads and the assembly built as it derives insights from known polymorphism and linkage disequilibrium in the target gene region.

In certain embodiments, the consensus sequence can be represented as a string of length n: S=S₁S₂ . . . Sₙ. The sequence reads can be represented using a matrix X of size n×m where n is the size of the genomic region and m is the number of sequence reads in the data: X=(Xᵢⱼ)_{m×n}. Each column in the matrix shows the overlap of the reads at position j along the genomic region and is represented by X.ⱼ. The posterior probability of the consensus sequence at position j given the matrix X is represented as πⱼ(a)=Pr(Sⱼ=a|X) where α is a nucleotide or an insertion or deletion. The set of possible values of a is represented as [symbol] ={A, C, G, T, −}. The base error rates are represented by p(b|a) and the prior distribution of nucleotide is represented by αₐ. The posterior probability can be calculated from the Bayesian model:

$${π}_{j} ( a ) = \frac{{α}_{a} {\prod }_{j = 1}^{n} p ( {X}_{ij} \vert a )}{{\sum }_{b ∈ 𝒜} {α}_{b} {\prod }_{j = 1}^{n} p ( {X}_{ij} \vert b )}$$

The base error rates can be determined from error model for the sequencing instrument and the quality of the basecall in each read. The prior distribution of the nucleotides can be determined from the reference sequences for the genomic region or from a neutral model where all nucleotides are equally likely.

In certain embodiments, the process of de novo assembly may be repeated after basecall assessment using Bayesian estimates and erroneous or unreliable sequence reads are eliminated.

The sequence read information is manipulated to identify polymorphic sites within the reads. For each position in the genetic locus, the probabilities of the underlying genotype are estimated by Bayesian model for the given sequence reads. The major allele is defined as the most frequent nucleotide at current position and minor allele is defined as the second most frequent nucleotide. The Minor Allele Frequency (MAF) is calculated as the frequency of the minor allele over all bases and gaps. Polymorphic sites are determined by applying thresholds on both genotype probability and MAF. From this analysis, a consensus sequence from the genotype calls at each position is generated. For a heterozygous genotype with multiple polymorphic sites, a dynamic programming algorithm is employed to construct two phased haplotype sequences, each representing one allele of the genetic locus.

After identification of polymorphic sites, the method processes the polymorphic site information into a polyread which excludes the conserved sequences that are present in the sequence reads and only retains the polymorphic site information. The reads from only the polymorphic sites are stored in an assembly matrix X The assembly matrix X has size m×n where m is the number of reads and n is the number of polymorphic sites. For instance, the sequence read information may be converted into a binary string comprising a value of 0 or 1 for each polymorphic site within said polyread. In some instances, the polyread is represented as Xᵢϵ{0,1,−}ⁿ, where “−” is a gap indicating that the position is not covered by the polyread. Some polyreads may not comprise a gap at either end of the polyread. While the values 0 and 1 are typically used for representing a binary string, other types of binary representation of the polyread can also be used.

A scoring matrix can be constructed to infer the haplotypes that minimize the discrepancy between the assignment and the reduced assembly matrix. In one embodiment, the method defines the haplotype sequence as binary string h consisting of 0 and 1, the complete haplotype has length n. Further, the method defines r as a k-mer of binary string and s(i, r) as the score for partial haplotypes starting at position 0 and ending at position i+k−1 with suffix r, where the partial haplotypes are the prefixes of full-length haplotypes. The dynamic programming algorithm then takes the reduced assembly matrix and constructs a scoring matrix by the following algorithm:

s(i,r)=min_{b=0,1}(s(i−1,(b,r[0,k−2]))+h(i,r)) (1)

where b is a binary number of 0 or 1, r[0, k−2] is the length k−1 prefix of r, (b, r[0,k−2]) is a k-mer binary string generated by concatenating b with r[0,k−2]), h(i, r) is the minimum of the total number of discrepancy between r or the complement of r and all reads starting at position i. For h(i,r), the method considers both r and the complement of r because each read can be assigned to either the current haplotype or its complement to minimize the discrepancy. By applying formula (1), one of the assignments with the minimum discrepancy is chosen and b is recorded for each entry in the matrix for trace back. Each gap “−” is ignored and a mismatch can only happen between 0 and 1 symbols. The first column of the scoring matrix s(0,r) can be initialized by considering the reads that start at position 0. For each read and r, compute the number of mismatches between the read and r and the read and the complement of r. And then add the minimum number to s(0,r). The partial haplotypes at position i can be obtained by extending the partial haplotypes at position i−1 with either a 0 or 1.

After the scoring matrix is computed, the algorithm can trace back to reconstruct the haplotypes that minimize the discrepancy. Starting from the solution rₙ that leads to a minimal value of s(n−k, r) over all r, the previous step is s(n−k−1, (b,rₙ[0, k−2])), where b is the recorded symbol for computing s(n−k, rₙ). Thus we obtain a partial haplotype (b, rₙ) starting at position n−k−1. By repeating the procedure until trace back to the first column of the scoring matrix, we can construct the complete haplotype starting at position 0. The time complexity of the dynamic programming algorithm is o(m×2^{k}×n), where m is the number of polyreads, k is the length of the longest polyread and n is the total number of polymorphic sites in the haplotypes.

In certain embodiments, the invention further provides a weighted scoring scheme at each position in the genetic locus to obtain a weighted score that minimizes the total number of discrepancies, wherein the weighted scoring scheme applies a weight in accordance with the quality of sequencing reads at each position. Weighted scoring scheme corrects for the errors introduced in the sequencing process. Each basecall in the sequencing reads is assigned a quality score, which can be interpreted as sequencing error for that basecall. For quality score q from Sanger or Illumina sequencing platform, the error probability can be derived as:

$$Pr ( Error ) = {10}^{- \frac{q - K}{10}}$$

where K is a constant number and q is greater or equal to K. For each discrepancy in the equation (1) above, the weight is (1−Pr(Error)) in the weighted scheme which is summed instead of 1 (which corresponds to an un-weighted scheme). Then h(i, r) can be defined as the minimum of the sum of the weighted discrepancy between r or the complement of r and all the reads starting at position i.

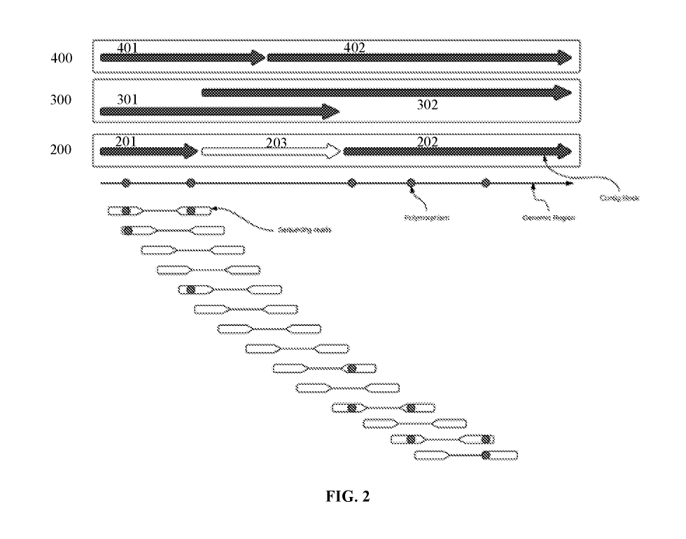

In certain embodiments, the assembly matrix can be broke down into subsets where there are no more than a certain number n₀ of polyreads that span any two sets. For example, in practice, n₀ can be chosen as an integer between 2 and 20 for the human HLA genes although a larger number can be used for less complicated genes. The dynamic programming algorithm can be applied to each subset independently and haplotype blocks can be constructed from each subset and then concatenated or ligated to produce the full-length haplotypes. Certain methods for partitioning the assembly matrix and constructing haplotype blocks are illustrated in FIG. 2.

Referring to FIG. 2, the partition of the assembly matrix into subsets for obtaining haplotype blocks can be done in a number of different ways. In a first partition 200, a haplotype block starts at the first polymorphic site in the subset 201 or the beginning of the genomic region and ends at the last polymorphic site in the subset 201 or the end of the genomic region for the last subset 202. The last polymorphic site in subset 201 and the first polymorphic site in the adjacent subset 202 define the boundaries of a homozygous region 203. In a second partition 300, the homozygous region can be merged into the left haplotype block defined by the subset 201 in the first partition 200 to give an extended haplotype block 301. The homozygous region can also be merged into the right haplotype block defined by the subset 202 in the first partition 200 on the right to give an extended haplotype block 302. In a third partition 400, the homozygous region defined in the second partition 300 can be broken down into two parts from the middle point or one of the end points. The left part can be merged into the haplotype block defined by the subset 201 in the first partition 200 to give an extended haplotype block 401 and the right part can be merged into the haplotype block defined by the subset 202 in the first partition 200 to give an extended haplotype block 402. One of ordinary skill in the art would understand that any other suitable partitions of the assembly matrix may be used for constructing haplotype blocks in the present invention.

Within each subset, two distinct consensus sequences, i.e., one pair of haplotypes, are constructed for the haplotype block. For n haplotype blocks, 2ⁿ⁻¹ unique pairs of complete haplotypes can be constructed by concatenating one of the two distinct consensus sequences from each of the n haplotype blocks to form a complete haplotype. The 2ⁿ⁻¹ unique pairs of complete haplotypes can then be compared to the reference sequences in the database to find the best matching reference pairs. The best matching reference pairs can be determined based on the number of mismatches in the alignment between the complete haplotype sequences to the reference sequences on the entire genomic region or a subset of the genomic region that is of particular interest. For example, the subset of the genomic region can be all the exon regions. The best matching reference pairs can also be determined based on an alignment score between the complete haplotype sequences and the reference sequences. The alignment score can measure either the similarity of the sequences or the distance of the sequences. The score can be chosen from the comparison of pairs of complete haplotypes and pairs of reference sequences by combining the numbers of mismatches in the alignment and the alignment score between a pair of complete haplotypes and a pair of reference sequences.

By partitioning the assembly matrix into subsets and estimating haplotypes within each subset first, the computational burden of the phasing analysis can be significantly reduced because the number of possible haplotypes increases exponentially with the number of polymorphic sites. The partition-ligation method provided herein is an implementation of the divide-and-conquer strategy and is particularly useful in phasing analysis of the human HLA genes. This is because the HLA gene region is long and highly polymorphic and the number of full-length haplotypes is extremely large. However, there exists strong linkage disequilibrium within certain regions of the HLA genes and therefore the number of haplotypes within each haplotype block can be significantly reduced when partial-haplotypes are constructed first. In certain embodiments, the partition-ligation method are useful in haplotype determination in the human HLA genes where the genetic locus has at least 25, at least 50, at least 75, at least 100 or even more polymorphic sites.

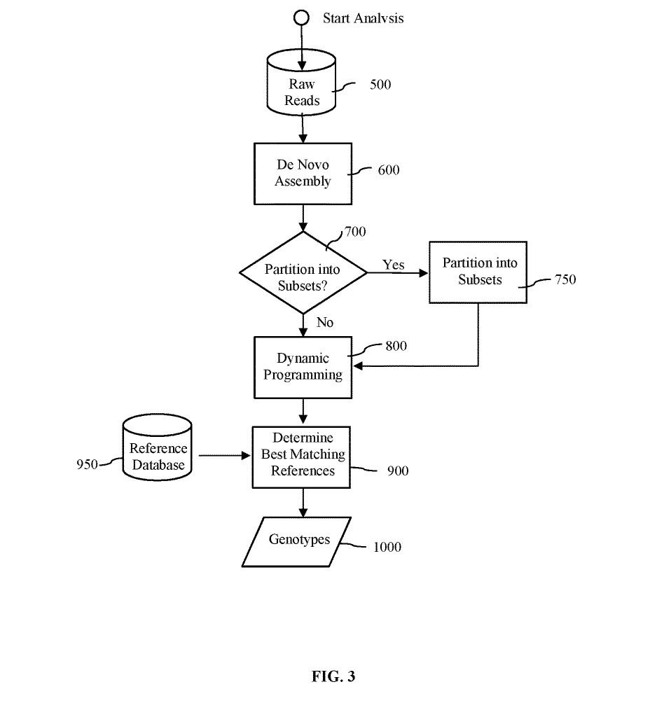

FIG. 3 illustrates an exemplary workflow in one embodiment of the present invention suitable for an improved phasing analysis of the human HLA genes using NGS data. The embodiment is implemented in the Mia Fora™ software system (Sirona Genomics, Palo Alto, Calif.). The workflow starts at step 500 by obtaining raw sequence reads from the NGS data. At step 600, the raw sequence reads are built into a de novo assembly read. The raw sequence reads are aligned to the assembly read using the Smith-Waterman algorithm to provide an assembly matrix. The assembly matrix is processed to produce a polyread containing only polymorphic sites in the data. At step 700, a decision is made as to whether the polyread should be partitioned into subsets and construct partial haplotypes within each subset first. If the decision is “Yes,” the polyread is partitioned at step 750 and within each subset, the dynamic programming algorithm is used in step 800 to obtain the optimal partial haplotype pairs that minimize the discrepancies between the haplotype pairs and the sequence reads. These partial haplotype pairs are then assembled into full length haplotypes. If the decision is “No” at step 700, the dynamic programming algorithm is applied to the entire polyread to obtain the optimal haplotype pairs at step 800. After the two phased haplotypes are obtained, the sequences are compared against a HLA gene reference database 950 at step 900 to determine the best matching references. At step 1000, the best matching reference are reported as the HLA genotype for a given sample. The next best matching references may also be reported together with the best one for a user to review, evaluate, and make a manual determination of the genotype. The workflow is validated through independent verification of the genotype calls using trio data comprising approximately 1,500 parents-offspring trios for human HLA genes HLA-A, HLA-B, HLA-C, HLA-DRB1, HLA-DRB3, HLA-DRB4, HLA-DRB5, HLA-DQB1, HLA-DQA1, HLA-DPB1, and HLA-DPA1. The results of the validation are shown in Table 1 and HLA-DRB3, HLA-DRB4 and HLA-DRB5 together are considered one locus in this validation. The validated accuracy for major HLA loci at 6 digit resolution is 98.8% using fully automated methods provided in the software. When results provided by the software are further reviewed and corrected, the accuracy of HLA genotype call is 99.8%. Further, concordance comparison of automatic and reviewed calls indicates that less than 1% of the calls require manual review and correction.

TABLE 1

| | Overall | | | | | | | |
|---|---|---|---|---|---|---|---|---|
| | | | | Class II | | | | |
| | Class I | | | HLA- | HLA- | HLA- | HLA- | HLA- |
| Locus | HLA-A | HLA-B | HLA-C | DRB1 | DRB3/4/5 | DQA1 | DQB1 | DPA1 |
| Automatic | 99.5% | 98.2% | 99.2% | 95.8% | 98.6% | 99.1% | 99.8% | 100.0% |
| 98.8% | 99.0% | | | 98.7% | | | | |
| Reviewed | 99.9% | 99.8% | 99.9% | 99.6% | 99.6% | 100.0% | 100.0% | 100.0% |
| 99.8% | 99.86% | | | 99.8% | | | | |
| Concordance* | 99.8% | 99.4% | 99.9% | 97.5% | 99.3% | 99.2% | 99.9% | 100.0% |

| | | |
|---|---|---|
| 99.4% | 99.7% | 99.2% |

*Concordance between the automatic and reviewed calls including all 3 members in the family trio.

We claim:

1. A method for assigning a partial haplotype to a genetic locus comprising:
a. providing sequence reads for said genetic locus;
b. processing said sequence reads into an assembly read;
c. generating a consensus sequence from said assembly read comprising only polymorphic sites within said genetic locus to produce a polyread;
d. constructing a scoring matrix by converting said polyread into a binary string;
e. processing said scoring matrix by generating a score that minimizes the total number of discrepancies between the consensus sequence and said sequence reads at only said polymorphic sites; and
f. assigning said partial haplotype to said genetic locus by reconstructing said locus using the score from step (e).

2. The method of claim 1, wherein said sequence reads are paired-end sequence reads.

3. The method of claim 1, wherein said binary string comprises a value of 0 or 1 for each polymorphic site within said polyread.

4. The method of claim 1, wherein said polyread is represented as Xᵢϵ{0,1,−}ⁿ, where “−” indicates a gap in a position is not covered by the polyread.

5. The method of claim 4, wherein said polyread does not comprise a gap at either end of said polyread.

6. The method of claim 1, wherein said scoring matrix is represented by a k-mer of said binary string and s(i,r) as the score for a partial haplotype starting at position 0 and ending at position i+k−1 with suffix r.

7. The method of claim 6, wherein said s(i,r)=min_{b=0,1}(s(i−1, (b, r[0, k−2]))+h(i,r)) where b is a binary number of 0 or 1, r[0, k−2] is the length k−1 prefix of r, (b, r[0, k−2]) is a k-mer binary string generated by concatenating b with r[0, k−2], h(i,r) is the minimum of the total number of discrepancies between r or the complement of r and all reads starting at position i.

8. The method of claim 7, wherein r comprises either r or the complement of r.

9. The method of claim 7, wherein said result for s(i,r) represents the minimum error correction (“MEC”) score.

10. The method of claim 7, wherein said s(i,r) excludes gaps in the polyread.

11. The method of claim 7 further comprising said partial haplotype is generated by iteratively from the solution rₙ which represents a minimal value of s(n−k,r) over all r.

12. The method of claim 11 further comprising where s(n−k−1, (b,rₙ [0, k−2])), where b is the recorded symbol for computing s(n−k, rₙ).

13. The method of claim 12, further comprising obtaining a partial haplotype (b,rₙ) iteratively from position n−k−1.

14. A method of generating a complete haplotype for a genetic locus by sequentially processing partial haplotypes for said locus generated by the method according to any one of claims 1-13.

15. The method according to any one of claims 1-14, wherein said method performed on a digital computer.

16. The method according to any one of claims 1-15, wherein said genetic locus is an HLA locus.

17. The method of claim 16, wherein said genetic locus is selected from the group consisting of HLA-A, HLA-B, HLA-C, HLA-DRB1, HLA-DRB3, HLA-DRB4, HLA-DRB5, HLA-DQB1, HLA-DQA1, HLA-DPB1, and HLA-DPA1.

18. The method of claim 1, wherein said method employs a Bayesian model in identifying said polymorphic sites.

19. The method of claim 1, wherein said method employs a minor allele frequency determination comprising assessing the frequency of the 2^{nd} most abundant base at a polymorphic site in said locus.

20. The method of claim 19, wherein step (b) further comprises generating the consensus sequence using a threshold cutoff of minor allele frequency.

21. The method of claim 1, wherein said locus comprises at least 10 polymorphic sites.

22. The method of claim 1, wherein said locus comprises at least 50 polymorphic sites.

23. The method of claim 1, further comprising between step (c) and step (d):
(c1) performing Bayesian estimates for at least one sequence read on said assembly read; and
(c2) adjusting said at least one sequence read and said polyread based on the result of said Bayesian estimates.

24. The method of claim 1, wherein said score in step (e) is a weighted score and wherein a weight is assigned to each position in the polyread based on a quality measurement of said position.

25. A method for assigning a haplotype to a genetic locus comprising:
a. providing sequence reads for said genetic locus;
b. processing said sequence data into an assembly read;
c. generating a consensus sequence from said assembly read comprising only polymorphic sites within said genetic locus to produce a polyread;
d. partitioning said polyread into at least two subsets, wherein each subset comprises at least two polymorphic sites;
e. obtaining, for each subset, a pair of partial haplotypes by
i. constructing a scoring matrix by converting said polyread into a binary string;
ii. processing said scoring matrix by generating a score that minimizes the total number of discrepancies between the consensus sequence and said sequence reads at only said polymorphic sites;
iii. assigning said partial haplotype to said genetic locus by reconstructing said locus using the score from step (i);
f. concatenating each partial haplotype from each subset with all other partial haplotypes from all other subsets to produce a collection of haplotype pairs spanning all polymorphic sites within the gene locus; and
g. assigning a haplotype pair to the genetic locus from said collection of haplotype pairs, wherein said haplotype pair minimizes the discrepancies between the consensus sequence and said sequence reads.

26. The method of claim 25, wherein said genetic locus is an HLA locus.

27. The method of claim 26, wherein said genetic locus is selected from the group consisting of HLA-A, HLA-B, HLA-C, HLA-DRB1, HLA-DRB3, HLA-DRB4, HLA-DRB5, HLA-DQB1, HLA-DQA1, HLA-DPB1, and HLA-DPA1.

28. A data processing system for generating a partial haplotype for a genetic locus comprising:
a. a digital computer with processing and information storage capabilities; and
b. a processing system for assigning at least one partial haplotype to a genetic locus, wherein said processing system is capable of performing the method according to any one of claims 1-27.

29. The method of claim 28, wherein said genetic locus is an HLA locus.

30. The method of claim 29, wherein said genetic locus is selected from the group consisting of HLA-A, HLA-B, HLA-C, HLA-DRB1, HLA-DRB3, HLA-DRB4, HLA-DRB5, HLA-DQB1, HLA-DQA1, HLA-DPB1, and HLA-DPA1.
